(19) United States
(12) Patent Application Publication
(10) Pub. No.: US 2022/0270728 A1
(43) Pub. Date: Aug. 25, 2022

(54) PATIENT UNIQUE IDENTIFIER

(71) Applicant: ZEBRA TECHNOLOGIES CORPORATION, Lincolnshire, IL (US)

(72) Inventors: Nicole Daphne Tricoukes, Seaford, NY (US); Daniella Strat, Sound Beach, NY (US); Askold V. Strat, Sound Beach, NY (US); Matthew Louis Kowalski, Merrick, NY (US)

(21) Appl. No.: 17/183,141

(22) Filed: Feb. 23, 2021

(51) Int. Cl. G16H 10/65; A61B 5/15; G02B 27/01

(52) U.S. Cl. CPC G16H 10/65; A61B 5/150786; A61B 5/150793; G02B 27/017

(57) ABSTRACT

A patient unique identifier system is provided. An example system includes a database storing patient information; a radio-frequency identification (RFID) tag associated with a patient, the RFID tag storing an indication of an identification of the patient and encrypted biometric data captured by sensors of a wearable device worn by the patient; and an RFID reader device configured to: read the RFID tag and retrieve, based on reading the RFID tag, the indication of the identification of the patient and the encrypted biometric data without decrypting the encrypted biometric data; send the encrypted biometric data to a server to be decrypted and stored in the database; access patient information associated with stored in the database based on the retrieved indication of the identification of the patient; and provide the information associated with the patient to a user of the RFID reader device.

### BACKGROUND

In healthcare settings, such as hospitals, doctors' offices, clinics, etc., healthcare workers must access patient medical information in order to provide appropriate healthcare services to each patient. However, electronically looking up each individual patient's medical information can be cumbersome for healthcare providers, especially when the healthcare provider is using a mobile device, or is moving between examination rooms, hospital beds, or other locations to provide care to many patients in quick succession. Additionally, this patient medical information is typically private and must be kept secure to avoid breaching patient confidentiality regulations.

### SUMMARY

In an embodiment, the present invention is a system, comprising: a database storing patient information; a wearable device worn by a patient, including one or more sensors configured to capture biometric data associated with the patient, wherein the wearable device is configured to encrypt the captured biometric data associated with the patient and store the encrypted biometric data on a radio-frequency identification (RFID) tag associated with a patient, the RFID tag storing an indication of an identification of the patient; an RFID reader device configured to: read the RFID tag; retrieve, based on reading the RFID tag, the indication of the identification of the patient; retrieve, based on reading the RFID tag, the encrypted biometric data associated with the patient, wherein the RFID reader device is not configured to decrypt the encrypted biometric data; send the received encrypted biometric data to server to be decrypted and stored in a database storing patient information; access patient information stored in the database based on the retrieved indication of the identification of the patient; and provide the information associated with the patient to a user of the RFID reader device.

In a variation of this embodiment, the patient information stored in the database includes one or more of: medical history information, biometric data information, medical test result information, or contact information associated with one or more respective patients.

Additionally, in a variation of this embodiment, the indication of the identification of the patient is an identification number or a string of characters unique to the patient.

As another example, in a variation of this embodiment, the RFID reader device is a mobile computing device.

Additionally, in a variation of this embodiment, the RFID reader device is a head's up display (HUD) device.

As another example, in a variation of this embodiment, providing the information associated with the patient to the user of the RFID reader device includes one or more of: displaying the information or generating an audio indication of the information.

As still another example, in a variation of this embodiment, the RFID reader device is further configured to: receive, from the user, additional information associated with the patient; and send the additional information associated with the patient to the database to be stored.

In another embodiment, the present invention is a method, comprising: reading, by a radio-frequency identification (RFID) reader device, an RFID tag associated with a patient, the RFID tag storing an indication of an identification of the patient and encrypted biometric data captured by sensors of a wearable device worn by the patient; retrieving, by the RFID reader device, based on reading the RFID tag, the indication of the identification of the patient and the encrypted biometric data, wherein the RFID reader device is not configured to decrypt the encrypted biometric data; sending, by the RFID reader device, the received encrypted biometric data to a server to be decrypted and stored in a database storing patient information; accessing, by the RFID reader device, patient information stored in the database based on the indication of the identification of the patient; and providing, by the RFID reader device, the information associated with the patient to a user of the RFID reader device.

In a variation of this embodiment, the patient information stored in the database includes one or more of: medical history information, biometric data information, medical test result information, or contact information associated with one or more respective patient.

Additionally, in a variation of this embodiment, the indication of the identification of the patient is an identification number or a string of characters unique to the patient.

As another example, in a variation of this embodiment, the RFID reader device is a mobile computing device.

Additionally, in a variation of this embodiment, the RFID reader device is a head's up display (HUD) device.

As another example, in a variation of this embodiment, providing the information associated with the patient to the user of the RFID reader device includes one or more of: displaying the information or generating an audio indication of the information.

As still another example, in a variation of this embodiment, the method further includes receiving, by the RFID reader device, from the user, additional information associated with the patient; and sending, by the RFID reader device, the additional information associated with the patient to the database to be stored.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIG. 1 illustrates a block diagram of an example system including a logic circuit for implementing the example methods and/or operations described herein, including methods for securely accessing a patient's data based on reading a patient unique identifier (PUID) device associated with the patient.

FIG. 2 illustrates an example interaction between a healthcare provider and a patient, in which the healthcare provider uses a reader device to securely access a patient's data based on reading a PUID device associated with the patient.

FIG. 3 illustrates a block diagram of an example process as may be implemented by the system of FIG. 1, for implementing example methods and/or operations described herein, including methods for securely accessing a patient's data based on reading a PUID device associated with the patient.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

The present application provides a patient unique identifier (PUID) device, such as a radio frequency identification (RFID) tag or other computer readable tag, that facilitates the retrieval of patient medical information from a secure location. In some examples, the PUID device may be a wearable device, such a bracelet, an anklet, a necklace, a chest strap, etc., worn by a patient in a healthcare setting. The PUID may store an identification code associated with a patient, and a reader device used by a healthcare professional may retrieve the identification code stored by the PUID and use the identification code to pull private patient medical information from a database. Accordingly, the PUIDs themselves do not store private patient medical information, but a healthcare professional using the reader device can access patient information for patients associated with PUIDs that are read by the reader device based on identification codes stored by each PUID.

In some examples, the PUID device may be a “passive” PUID device. A passive PUID device may facilitate the retrieval of patient information without containing any local source of energy, such as a battery. For instance, a passive PUID may be a passive RFID tag, or it may contain information in a graphic form that can be read electronically, such as a barcode. For example, the passive PUID device may store an identification code that can be used to access entry to a database that contains patient information for the patient associated with the PUID device, but does not itself store any actual patient medical information. Accordingly, only a computing device (i.e., a reader device) that is able to access the database can access patient medical information using the identification code provided by the PUID device. Thus, the identification code stored in the PUID device need not be secure as it cannot be used to access the patient medical information from the database without a computing device that has security clearance to access the database.

Additionally, in some examples, the PUID device may be an “active” PUID device and may contain additional information about the patient that can be written to and read from the active PUID device, i.e., by the means of a radio transceiver in the case of RFID tags. An active PUID device may have its own power source and may be able to perform tasks autonomously. For instance, an active PUID device may be a medical monitoring device that is configured to monitor patient medical data, such as the patient's heart rate, temperature, breathing patterns, etc. As the device is worn by the patient, the active PUID device may monitor and record this information, which may be retrieved later using a reader device. Unlike a passive PUID, which is primarily used to associate a patient with the stored information from the database, the active PUID can capture new information about the patient which may in turn be added to the database.

The active PUID may keep the patient medical data private and secure by encrypting this data such that it cannot be retrieved directly by a reader device. That is, like the passive PUID device, the active PUID device may store an identification code associated with the patient. The reader device may retrieve the identification code from the active PUID device, along with any encrypted patient medical data associated with the patient that is stored by the active PUID device. The reader device may then send the identification code and the encrypted medical data to a server associated with the patient database. The encrypted patient medical data may be decrypted on the server side and then written to the patient database. The reader device may then access the newly stored information from the database using the identification code from the active PUID. The reader device may also update the patient database with additional patient medical data provided by the healthcare provider.

Accordingly, the reader does not need to be involved in the encryption or decryption of the secure patient medical data, but simply passes the encrypted patient medical data retrieved from the active PUID to the server that services the database. The encryption key may be stored on the server and may be written to the active PUID before being assigned to a patient such that it is not read from the PUID once assigned. In this way the PUID device uses the key to encrypt the stored data, which can only be decrypted by the server.

In any case, the PUID device and reader device system provides the necessary security and privacy for accessing and storing patient medical information. In some examples, a reader device that communicates with a PUID device may be a mobile computing device with RFID and barcode reading capability. For instance, in some examples, the reader device may be a hand-held device or a wearable computer, e.g., an intelligent head's up display.

Additionally, in some examples, the PUID device (either active or passive) may also be used to provide real-time patient location information, as well as tracking history, provided that the appropriate infrastructure is deployed in the monitored area. For example, when the PUID is an RFID tag, an infrastructure of RFID readers with adequate coverage could be installed at various locations throughout a hospital building or campus. Similarly, for the case of graphical PUID, a network of video cameras could be installed throughout the monitored area. For instance, this may be useful in understanding the evolution of a disease and preventing patients from coming into contact with or spreading pathogens.

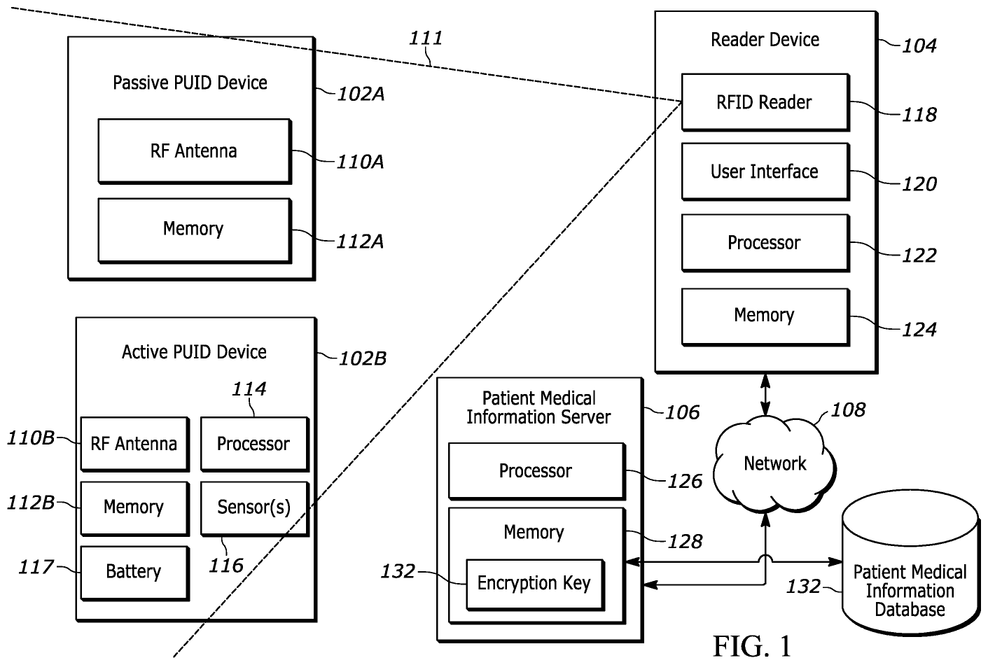

FIG. 1 illustrates a block diagram of an example system 100 including a logic circuit for implementing the example methods and/or operations described herein, including methods for securely accessing a patient's data based on reading a PUID device associated with the patient. The system 100 may include one or more passive PUID devices 102A and/or one or more active PUID devices 102B, each associated with a particular patient, as well as a reader device 104 configured to retrieve data from the PUID devices 102A/102B. For instance, the reader device 104 may retrieve a unique patient identification code from each PUID device 102A/102B. Additionally, in some examples, the reader device 104 may further receive encrypted patient medical data from the active PUID device 102B. The reader device 104 may communicate with a patient medical information server 106, e.g., via a wired or wireless network 108, in order to access patient medical data based on a patient identification code retrieved from the PUID devices 102A/102B, and/or in order to send encrypted patient medical data retrieved from the active PUID devices 102B.

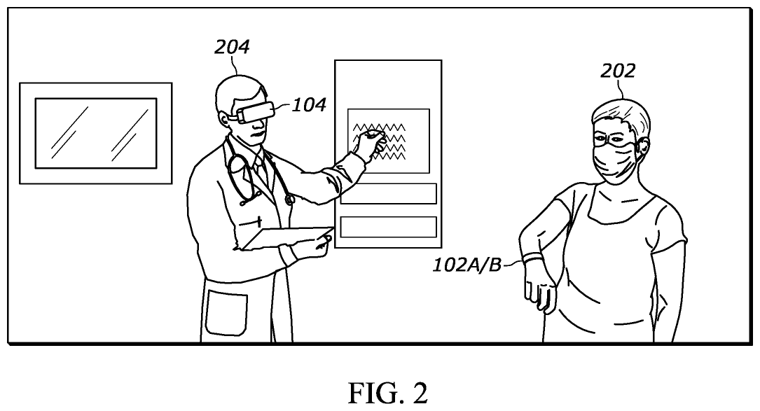

The passive PUID devices 102A and the active PUID devices 102B may be RFID tags implemented by wearable devices such as wristbands, armbands, necklaces, lanyards, anklets, rings, etc., worn by patients in a healthcare setting, such as a hospital. For example, FIG. 2 illustrates a patient 202 wearing a wristband implemented PUID device 102A/102B.

Referring back to FIG. 1, each passive PUID device 102A may include an RF antenna 110A, as well as a memory 112A storing an identification code for a patient associated with the passive PUID device 102A.

Similarly, each active PUID device 102B may include an RF antenna 110B, as well as a memory 112B storing an identification code for a patient associated with the active PUID device 102B. Each active PUID device 102B may further include one or more processors 114, one or more sensors 116, and a battery 117 configured to power the operations of the components of the active PUID device 102B. For instance, the sensors 116 may include various types of sensors configured to capture biometric data associated with the patient, such as, e.g., heart rate sensors, blood oxygen sensors, etc. Additionally, in some examples, the sensors 116 may include location sensors, such as GPS sensors. In addition to the patient identification code, the memory 112B (e.g., volatile memory, non-volatile memory) of each active PUID device 102B may further store data captured by the sensors 116 and may be accessible by the one or more processors 114 (e.g., via a memory controller). The one or more processors 114 may interact with the memory 112B to obtain, for example, computer-readable instructions stored in the memory 112B. The computer-readable instructions stored in the memory 112B may cause the one or more processors 116 to encrypt the patient biometric data captured by the sensors 116, and transmit the encrypted patient biometric data, along with the patient identification code 116, via the antenna 110B.

The reader device 104 may be a mobile computing device used by a healthcare professional such as a doctor or a nurse. For instance, the reader device 104 may be a smart phone, a tablet, or a wearable device such as a smart watch or a head's up display (HUD) device. For example, FIG. 2 illustrates a healthcare professional 104 wearing a HUD-implemented reader device 104. Referring back to FIG. 1, the reader device 104 may include an RFID reader 118 configured to retrieve data from the PUID devices 102A/102B, within its RFID range 111, via their respective antennas 110A/110B, as well as a user interface 120 configured to receive user input and provide information to the user (e.g., via a display screen or other visual display, via an audio output, etc.). Additionally, the reader device 104 may include one or more processors 122 and a memory 124 (e.g., volatile memory, non-volatile memory) accessible by the one or more processors 122 (e.g., via a memory controller). The one or more processors 122 may interact with the memory 124 to obtain, for example, computer-readable instructions stored in the memory 124.

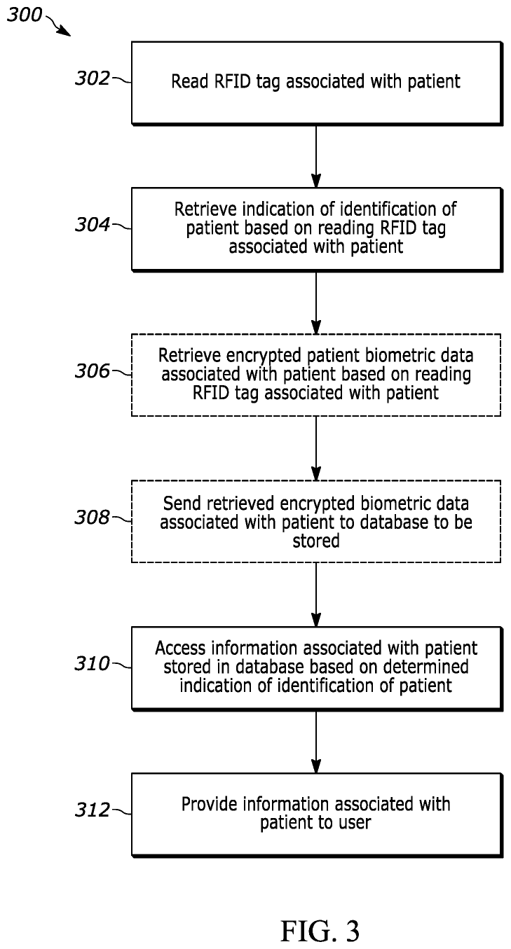

The computer-readable instructions stored in the memory 124 may cause the one or more processors 122 to communicate with the patient medical information server 106, e.g., via the network 108, to request patient medical information based on the patient identification codes obtained from the PUID devices 102A/102B. For example, each patient identification code may correspond to a particular patient, and the patient's medical information (or other identifying information) may be stored according to the patient identification code. Furthermore, the computer-readable instructions stored in the memory 124 may cause the one or more processors 122 to provide the accessed patient medical information from the patient medical information server 106 to a user of the reader device 104 via the user interface 120. Additionally, the computer-readable instructions stored in the memory 124 may cause the one or more processors 122 to send any encrypted patient biometric data retrieved from the active PUID devices 102B to the patient medical information server 106, e.g., via the network 108. Moreover, the computer-readable instructions stored in the memory 124 may cause the one or more processors 122 to send patient data received from the user of the reader device 104 via the user interface 120 to the patient medical information server 106, e.g., via the network 108. Furthermore, in some examples, the computer-readable instructions stored in the memory 124 may cause the one or more processors 122 to receive location data associated with various patients from the PUID devices 102A/102B associated with each patient and provide the locations of various patients to a user via the user interface 120. Additionally, the computer-readable instructions stored on the memory 124 may include instructions for carrying out any of the steps of the method 300, described in greater detail below with respect to FIG. 3.

The patient medical information server 106 may include one or more processors 126 and a memory 128 (e.g., volatile memory, non-volatile memory) accessible by the one or more processors 122 (e.g., via a memory controller). The one or more processors 126 may interact with the memory 128 to obtain, for example, computer-readable instructions stored in the memory 128, and/or an encryption key 130 stored in the memory 128. The computer-readable instructions stored in the memory 128 may cause the one or more processors 126 to send patient medical information, from the memory 128 and/or from patient medical information database 132, to the reader device 104 based on a patient identification code retrieved by the reader device 104. Additionally, the computer-readable instructions stored in the memory 128 may cause the one or more processors 126 to receive encrypted patient biometric data from the reader device 104, decrypt the encrypted biometric data using the encryption 130, and store the decrypted patient biometric data in the memory 128 and/or in the patient medical information database 132. Furthermore, in some examples, the computer-readable instructions stored in the memory 128 may cause the one or more processors 126 to receive patient medical information or other patient information (i.e., patient medical information or other patient information input by a user of the reader device 104, via the user interface 120) from the reader device 104, and store the patient medical information or other patient information in the memory 128 and/or in the patient medical information database 132.

FIG. 3 illustrates a block diagram of an example process as may be implemented by the system of FIG. 1, for securely accessing a patient's data based on reading a PUID device associated with the patient. One or more steps of the method 300 may be implemented as a set of instructions stored on a computer-readable memory (e.g., memory 122) and executable on one or more processors (e.g., processors 124).

At block 302, an RFID tag associated with a patient may be read by a radio-frequency identification (RFID) reader device, such as the reader device 104. The RFID tag may be associated with a wearable device worn by the patient and may store an indication of an identification of the patient. The indication of the identification of the patient may be an identification number or a string of characters unique to the patient, such as an identification code associated with the patient. Additionally, in some examples, the wearable device worn by the patient may further include one or more sensors configured to capture biometric data associated with the patient, and may be configured to encrypt the captured biometric data associated with the patient and store the encrypted biometric data on the RFID tag.

At block 304, the indication of the identification of the patient may be retrieved, e.g., by the RFID reader device based on reading the RFID tag. At block 306, optionally, the encrypted biometric data associated with the patient may be retrieved, e.g., by the RFID reader device based on reading the RFID tag. At block 308, optionally, the encrypted biometric data associated with the patient retrieved at block 306 may be sent, e.g., by the RFID reader, device to a patient medical information database to be stored.

Additionally, in some examples, the RFID reader device may receive additional information associated with a patient from a user, e.g., via a user interface of the RFID reader device, and may send this additional information to the patient medical information database.

At block 310, information associated with the patient may be accessed from the patient medical information database, e.g., by the RFID reader device, based on the indication of the identification of the patient. For instance, the database may store patient medical information (e.g., in some cases including decrypted biometric data sent to the database at block 308) and may include indications of which identification codes are associated with which patient medical information. The patient information stored in the database may include, e.g., medical history information, biometric data information, medical test result information, and/or contact information associated with each respective patient.

At block 312, information associated with the patient may be provided to a user of the RFID reader device, e.g., by a user interface of the RFID reader device that displays the information or generates an audio indication of the information. For instance, a healthcare provider using the RFID reader device may be able to view medical history information, biometric data information, medical test result information, and/or contact information associated with each respective patient after the RFID reader device retrieves the patient identification code from the RFID tag associated with the patient and accesses the patient information from the database using the patient identification code.

The above description refers to a block diagram of the accompanying drawings. Alternative implementations of the example represented by the block diagram includes one or more additional or alternative elements, processes and/or devices. Additionally or alternatively, one or more of the example blocks of the diagram may be combined, divided, re-arranged or omitted. Components represented by the blocks of the diagram are implemented by hardware, software, firmware, and/or any combination of hardware, software and/or firmware. In some examples, at least one of the components represented by the blocks is implemented by a logic circuit. As used herein, the term “logic circuit” is expressly defined as a physical device including at least one hardware component configured (e.g., via operation in accordance with a predetermined configuration and/or via execution of stored machine-readable instructions) to control one or more machines and/or perform operations of one or more machines. Examples of a logic circuit include one or more processors, one or more coprocessors, one or more microprocessors, one or more controllers, one or more digital signal processors (DSPs), one or more application specific integrated circuits (ASICs), one or more field programmable gate arrays (FPGAs), one or more microcontroller units (MCUs), one or more hardware accelerators, one or more special-purpose computer chips, and one or more system-on-a-chip (SoC) devices. Some example logic circuits, such as ASICs or FPGAs, are specifically configured hardware for performing operations (e.g., one or more of the operations described herein and represented by the flowcharts of this disclosure, if such are present). Some example logic circuits are hardware that executes machine-readable instructions to perform operations (e.g., one or more of the operations described herein and represented by the flowcharts of this disclosure, if such are present). Some example logic circuits include a combination of specifically configured hardware and hardware that executes machine-readable instructions. The above description refers to various operations described herein and flowcharts that may be appended hereto to illustrate the flow of those operations. Any such flowcharts are representative of example methods disclosed herein. In some examples, the methods represented by the flowcharts implement the apparatus represented by the block diagrams. Alternative implementations of example methods disclosed herein may include additional or alternative operations. Further, operations of alternative implementations of the methods disclosed herein may combined, divided, re-arranged or omitted. In some examples, the operations described herein are implemented by machine-readable instructions (e.g., software and/or firmware) stored on a medium (e.g., a tangible machine-readable medium) for execution by one or more logic circuits (e.g., processor(s)). In some examples, the operations described herein are implemented by one or more configurations of one or more specifically designed logic circuits (e.g., ASIC(s)). In some examples the operations described herein are implemented by a combination of specifically designed logic circuit(s) and machine-readable instructions stored on a medium (e.g., a tangible machine-readable medium) for execution by logic circuit(s).

As used herein, each of the terms “tangible machine-readable medium,” “non-transitory machine-readable medium” and “machine-readable storage device” is expressly defined as a storage medium (e.g., a platter of a hard disk drive, a digital versatile disc, a compact disc, flash memory, read-only memory, random-access memory, etc.) on which machine-readable instructions (e.g., program code in the form of, for example, software and/or firmware) are stored for any suitable duration of time (e.g., permanently, for an extended period of time (e.g., while a program associated with the machine-readable instructions is executing), and/or a short period of time (e.g., while the machine-readable instructions are cached and/or during a buffering process)). Further, as used herein, each of the terms “tangible machine-readable medium,” “non-transitory machine-readable medium” and “machine-readable storage device” is expressly defined to exclude propagating signals. That is, as used in any claim of this patent, none of the terms “tangible machine-readable medium,” “non-transitory machine-readable medium,” and “machine-readable storage device” can be read to be implemented by a propagating signal.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. Additionally, the described embodiments/examples/implementations should not be interpreted as mutually exclusive and should instead be understood as potentially combinable if such combinations are permissive in any way. In other words, any feature disclosed in any of the aforementioned embodiments/examples/implementations may be included in any of the other aforementioned embodiments/examples/implementations.

The benefits, advantages, solutions to problems, and any element(s) that may cause any benefit, advantage, or solution to occur or become more pronounced are not to be construed as a critical, required, or essential features or elements of any or all the claims. The claimed invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separately claimed subject matter.

1. A system, comprising:
a database storing patient information;
a wearable device worn by a patient, including one or more sensors configured to capture biometric data associated with the patient, wherein the wearable device is configured to encrypt the captured biometric data associated with the patient and store the encrypted biometric data on a radio-frequency identification (RFID) tag associated with a patient, the RFID tag storing an indication of an identification of the patient;
an RFID reader device configured to:
read the RFID tag;
retrieve, based on reading the RFID tag, the indication of the identification of the patient;
retrieve, based on reading the RFID tag, the encrypted biometric data associated with the patient, wherein the RFID reader device is not configured to decrypt the encrypted biometric data;
send the received encrypted biometric data to server to be decrypted and stored in a database storing patient information;
access patient information stored in the database based on the retrieved indication of the identification of the patient; and
provide the information associated with the patient to a user of the RFID reader device.

2. The system of claim 1, wherein the patient information stored in the database includes one or more of: medical history information, biometric data information, medical test result information, or contact information associated with one or more respective patient.

3. The system of claim 1, wherein the indication of the identification of the patient is an identification number or a string of characters unique to the patient.

4. The system of claim 1, wherein the RFID reader device is a mobile computing device.

5. The system of claim 1, wherein the RFID reader device is a head's up display (HUD) device.

6. The system of claim 1, wherein providing the information associated with the patient to the user of the RFID reader device includes one or more of: displaying the information or generating an audio indication of the information.

7. The system of claim 1, wherein the RFID reader device is further configured to:
receive, from the user, additional information associated with the patient; and
send the additional information associated with the patient to the database to be stored.

8. A method, comprising:
reading, by a radio-frequency identification (RFID) reader device, an RFID tag associated with a patient, the RFID tag storing an indication of an identification of the patient and encrypted biometric data captured by sensors of a wearable device worn by the patient;
retrieving, by the RFID reader device, based on reading the RFID tag, the indication of the identification of the patient and the encrypted biometric data, wherein the RFID reader device is not configured to decrypt the encrypted biometric data;sending, by the RFID reader device, the received encrypted biometric data to a server to be decrypted and stored in a database storing patient information;
accessing, by the RFID reader device, patient information stored in the database based on the indication of the identification of the patient; and
providing, by the RFID reader device, the information associated with the patient to a user of the RFID reader device.

9. The method of claim 8, wherein the patient information stored in the database includes one or more of: medical history information, biometric data information, medical test result information, or contact information associated with one or more respective patient.

10. The method of claim 8, wherein the indication of the identification of the patient is an identification number or a string of characters unique to the patient.

11. The method of claim 8, wherein the RFID reader device is a mobile computing device.

12. The method of claim 8, wherein the RFID reader device is a heads up display (HUD) device.

13. The method of claim 8, wherein providing the information associated with the patient to the user of the RFID reader device includes one or more of: displaying the information or generating an audio indication of the information.

14. The method of claim 8, further comprising:
receiving, by the RFID reader device, from the user, additional information associated with the patient; and
sending, by the RFID reader device, the additional information associated with the patient to the database to be stored.
